(19) United States
(12) Patent Application Publication
(10) Pub. No.: US 2008/0318910 A1
(43) Pub. Date: Dec. 25, 2008

(54) Controlled-Release Oral Dosage Form

(75) Inventors: Alain Desjardins, Montreal (CA); Bertrand Bolduc, Longueuil (CA)

Correspondence Address: BERESKIN AND PARR, 40 KING STREET WEST, BOX 401, TORONTO, ON, M5H 3Y2, CA

(73) Assignee: MISTRAL PHARMA, INC., Dorval, QC (CA)

(21) Appl. No.: 12/089,068

(22) PCT Filed: Oct. 3, 2006
(86) PCT No.: PCT/CA2006/001633

§ 371 Date: Apr. 24, 2008

Related U.S. Application Data
(60) Provisional application No. 60/722,971, filed on Oct. 4, 2005.

(51) Int. Cl. A61K 31/65; A61P 1/00; A61P 17/10

(52) U.S. Cl. 514/152

(57) ABSTRACT

A controlled-release oral dosage form suitable for administration to a mammal. The dosage form comprises a drug and an organic acid or a salt thereof. The dosage form releases no more than 60% the total amount of the drug within about a first hour, the remaining amount of the drug being released over a period of time of about 5 to about 8 hours subsequent to the first hour. Such a dosage form can be particularly useful for delivery of various pH-dependent solubility drugs.

### FIELD OF THE INVENTION

The present invention relates to an oral dosage form. In particular, the invention relates to controlled-release oral dosage which is suitable for various drugs.

### BACKGROUND OF THE INVENTION

In the field of controlled-release drugs several solutions have been proposed in the last decade. However, there is still some room for improvement with respect to the delivery of various drugs, such as for drugs having a pH-dependent solubility. Many drugs have very different solubilities depending on the pH of the environment into which they are. As a consequence, these drugs will have dissolution rates, and therefore release rates from release devices, which will be different in stomach than in intestine, unless the release device is designed to compensate for the variability in release rate with pH.

A second difficulty in developing a once-a-day dosage form for a pH-dependent solubility drug is that some molecules have in addition a short absorption window, being mainly absorbed from the upper part of the small intestine, as indicated in U.S. Pat. No. 5,262,173 for minocycline, for example. In order to obtain a satisfactory once-a-day product, it is highly desirable to extend the residence time of the dosage form in the gastro-intestinal region where absorption is optimal. One of the previously proposed ways to extend retention of a drug delivery device in the upper gastro-intestinal region was to retain it in the stomach. This can be achieved by the use of a large diameter floating device, ingested along with the main meal of the day (Hou et al. Critical Reviews™ in Therapeutic Drug Carrier Systems, 20(6), 461-497, 2003). A large floating tablet is retained in the stomach after a meal till after the content of the stomach is emptied to the intestine at the end of the digestive cycle. Using this approach, the transit time in stomach and small intestine can be extended up to 6 to 10 hours.

As previously indicated, a major difficulty encountered in drug delivery is the variation of the solubility of a drug as a function of pH. The difficulty of developing a controlled-release dosage form with molecules having a pH-dependant solubility has been the subject of many publications.

Some authors have used high molecular weight polyacids to offset the reduced solubility of the drug as pH increases. The polyacids become soluble at higher pH, which triggers erosion of the system. The rapid erosion of the system counter-balances the decreased solubility of the drug.

WO 97/18814 describes a formulation comprising a low molecular weight polyethylene oxide, HPMC and one or more enteric polymers such as methacrylic acid copolymers. With compounds being more soluble at low pH value, the drug release from the tablet in the stomach is reduced by the enteric polymer. However, as the device travel along the gastrointestinal tract and the pH increases, the erosion rate increases to compensate for the reduced compound solubility.

U.S. Pat. No. 4,968,508 describes a formulation comprising a hydrophilic polymer (e.g. HPMC) and an enteric polymer (e.g. Eudragit L-100-55). In acidic medium, the enteric polymer is insoluble and acts as a part of the matrix and thus contributes to the retardation of the drug release. At higher pH-values, the enteric polymer dissolves and thus increases the permeability of the dosage form.

U.S. Pat. No. 4,792,452 describes a formulation containing an anionic (sodium alginate) and non-ionic polymer (HPMC). At low pH values, sodium alginate precipitates in the hydrated gel layer as alginic acid and provides resistance against erosion. At higher pH-values, the alginate forms a soluble salt, thus providing less resistance to erosion. The release mechanism changes from predominantly diffusion-controlled to predominantly erosion-controlled. The resulting higher permeability of the gel layer at high pH-value compensates the lower drug solubility, leading to a constant drug release rates at different pH-values.

The approaches described above would be well suited to release minocycline independently of surrounding's pH, but would fail to extend the residence time of minocycline in the gastrointestinal region.

In other cases low molecular weight acids in “reservoir” systems have been used. The matrix tablet or film coated device acts as a reservoir where the organic acid keeps internal pH low, even when the device is in high pH medium.

Kranz et al. in Pharmaceutical Sciences “Development of a single unit extended release formulation for ZK 811 752, a weakly basic drug” (2005, article in press) describes the use of organic acids such as fumaric, tartaric, adipic, glutamic and sorbic in combination with matrix former polymers like Kollidon SR, ethylcellulose, or HPMC. The addition of organic acids to all three matrix formers was found to maintain low pH-values within the tablets in phosphate buffer pH 6.8.

Streubel et al. in Journal of Controlled Release, 67 (2000) 101-110, describe a pH-independent release of verapamil HCl from matrix tablets consisting of ethylcellulose or HPMC by the addition of fumaric, sorbic or adipic acid. They demonstrated that the addition of organic acids to both matrix formers maintained low pH-values within the tablets during drug release in phosphate buffer pH 6.8.

Gabr in Eur. J. Pharm. Biopharm, 38(6) (1992) 199-202 studied the effect of citric, tartaric, and succinic acid on the drug release from matrix tablets consisting of cellulose acetate and beeswax as matrix former in various ratio.

The “reservoir” systems described above present several drawbacks. One of them is the particular shape of the release profile obtained from reservoir systems: the release rate decreases progressively leading to a first order release profile, often described as an exponential decay. The desired release profile for minocycline or other molecules cannot always be met by such systems. Moreover, for drug with short absorption windows, the likelihood of lower bioavailability is high as the last 25% of the drug is released very slowly. Finally, such systems but would fail to extend the residence time of minocycline in the gastrointestinal region.

It would therefore be highly desirable to be provided with a controlled-release dosage form that permit to overcome the prior art drawbacks.

### SUMMARY OF THE INVENTION

In accordance with one aspect of the present invention, there is provided a controlled-release oral dosage form suitable for administration to a mammal. The dosage form comprises:

a drug and sorbic acid or a salt thereof,

wherein the dosage form releases no more than 60% of the total amount of the drug within about a first hour, the remaining amount of the drug being released over a period of time of about 5 to about 8 hours subsequent to the first hour.

In accordance with another aspect of the present invention there is provided a controlled-release oral dosage form comprising:

a drug and sorbic acid or a salt thereof,

wherein the sorbic acid reduces the dissolution rate of the drug at a pH of about 1.0 to about 3.5, and enhances the dissolution rate of the drug at a pH of about 4.0 to about 7.0.

In accordance with another aspect of the present invention there is provided a controlled-release oral dosage form comprising:

a drug and an organic acid or a salt thereof, the organic acid having a solubility lower than 20 mg/mL (preferably lower than 10 mg/mL, and more preferably lower than 3.0 mg/mL) at a pH below 5.0 and a solubility of at least 100 mg/mL (preferably at least 200 mg/mL and more preferably at least 300 mg/mL) at a pH higher than 5.0,

wherein the organic acid or salt thereof is effective for reducing the dissolution rate of the drug at a pH of about 1.0 to about 3.5, and enhancing the dissolution rate of the drug at a pH of about 4.0 to about 7.0. In accordance with another aspect of the present invention there is provided a controlled-release oral dosage form comprising:

a drug and an organic acid or a salt thereof, the organic acid having a solubility lower than 20 mg/mL (preferably lower than 10 mg/mL, and more preferably lower than 3.0 mg/mL) at a pH below 5.0 and a solubility of at least 100 mg/mL (preferably at least 200 mg/mL and more preferably at least 300 mg/mL) at a pH higher than 5.0,

wherein the organic acid or salt thereof is effective for maintaining the dissolution rate of the drug at a value of about 5% by weight per hour to about 50% by weight per hour, based on the total weight of the drug included in the dosage form, when the dosage form is at a pH of about 4.0 to about 7.0.

In accordance with another aspect of the present invention there is provided a controlled-release oral dosage form comprising:

a drug and an organic acid or a salt thereof, the organic acid having a solubility lower than 20 mg/mL (preferably lower than 10 mg/mL, and more preferably lower than 3.0 mg/mL) at a pH below 5.0 and a solubility of at least 100 mg/mL (preferably at least 200 mg/mL and more preferably at least 300 mg/mL) at a pH higher than 5.0,

wherein the organic acid or salt thereof is effective for maintaining the dissolution rate of the drug at a value of about 10% by weight per hour to about 60% by weight per hour, based on the total weight of the drug included in the dosage form, when the dosage form is at a pH of about 1.0 to about 3.5.

In accordance with another aspect of the present invention there is provided a controlled-release oral dosage form suitable for human administration comprising:

a drug and an organic acid or a salt thereof, the organic acid having a solubility lower than 20 mg/mL (preferably lower than 10 mg/mL, and more preferably lower than 3.0 mg/mL) at a pH below 5.0 and a solubility of at least 100 mg/mL (preferably at least 200 mg/mL and more preferably at least 300 mg/mL) at a pH higher than 5.0,

wherein the dosage form releases no more than 60% of the total amount of the drug within a first hour, the remaining amount of the drug being released over a period of time of about 5 to about 8 hours subsequent to the first hour.

In accordance with another aspect of the present invention there is provided a controlled-release oral dosage form comprising:

a drug and an organic acid selected form the group consisting of fumaric acid, adipic acid, glutamic acid, sorbic acid, benzoic acid and salts thereof,

wherein the organic acid or salt thereof is effective for maintaining the dissolution rate of the drug at a value of about 5% by weight per hour to about 50% by weight per hour, based on the total weight of the drug included in the dosage form, when the dosage form is at a pH of about 4.0 to about 7.0.

In accordance with another aspect of the present invention there is provided a controlled-release oral dosage form comprising:

a drug and an organic acid selected form the group consisting of fumaric acid, adipic acid, glutamic acid, sorbic acid, benzoic acid and salts thereof,

wherein the organic acid or salt thereof is effective for maintaining the dissolution rate of the drug at a value of about 10% by weight per hour to about 60% by weight per hour, based on the total weight of the drug included in the dosage form, when the dosage form is at a pH of about 1.0 to about 3.5.

In accordance with another aspect of the present invention there is provided controlled-release oral dosage form comprising:

a drug, and an organic acid selected form the group consisting of fumaric acid, adipic acid, glutamic acid, sorbic acid, benzoic acid and salts thereof,

wherein the dosage form releases no more than 60% of the total amount of the drug within a first hour, the remaining amount of the drug being released over a period of time of about 5 to about 8 hours subsequent to the first hour.

In accordance with another aspect of the present invention there is provided a controlled-release oral dosage form comprising:

a drug and an organic acid selected form the group consisting of fumaric acid, adipic acid glutamic acid, sorbic acid, and salts thereof

wherein the organic acid or salt thereof reduces the dissolution rate of the drug at a pH of about 1.0 to about 3.5, and enhances the dissolution rate of the drug at a pH of about 4.0 to about 7.0.

In accordance with another aspect of the present invention there is provided a controlled-release oral dosage form comprising:

a drug and an organic acid selected form the group consisting of fumaric acid, adipic acid, glutamic acid, sorbic acid, benzoic acid and salts thereof

wherein the drug in the dosage form has a dissolution rate of about 10% by weight per hour to about 60% by weight per hour when being at a pH of about 1.0 to about 3.5, and a dissolution rate of about 5% by weight per hour to about 50% by weight per hour when being at a pH of about 4.0 to about 7.0.

In accordance with another aspect of the present invention there is provided a controlled-release oral dosage form comprising:

a drug and an organic acid selected form the group consisting of fumaric acid, adipic acid, glutamic acid, sorbic acid, benzoic acid and salts thereof

wherein the drug in the dosage form has a dissolution rate which is substantially the same when submitted to a pH of about 1.0 to about 3.5 and when submitted to a pH of about 4.0 to about 7.0.

It was found that the dosage forms of the present invention were very efficient for the drug delivery of pH-dependent solubility drugs. It was also found that such dosage forms were particularly useful for drugs having a high solubility at pH of about 1.0 to about 3.5 (pH of stomach) and a lower solubility at pH of about 4.0 to about 7.0 (pH of intestine). In fact, the organic acids present in these dosage forms permitted to reduce the dissolution rate of the drug at pH of about 1.0 to about 3.5, and to enhance the dissolution rate of the drug at a pH of about 4.0 to about 7.0.

For the purpose of the present invention the following terms are defined below.

The expression “pH-dependent solubility drug” as used herein refers to a drug which has a dissolution rate that will vary in accordance with pH. In fact, such a drug will have as example a solubility which will be different at pH of 1.0 to 3.5 (pH of stomach) than at pH 4.0 to 7.0 (pH of intestine).

The expression “pH insensitive” as uses herein refers to a condition so that when comparing the percent released under various pH conditions, the percent released for any given time point does not vary by more than 30% preferably less than 20%, most preferably less than 15%.

In the dosage forms of the present invention the drug is preferably a pH-dependent solubility drug. Preferably, such a drug is substantially soluble at a first pH of about 1.0 to about 3.5 and substantially less soluble at a pH of about 4.0 to 7.0 than at the first pH. The drug can be selected from the group consisting of doxycycline, tetracycline, oxytetracycline, minocycline, chlortetracycline, trimebutine, demeclocycline and salts thereof. Preferably, the drug is minocycline hydrochloride or trimebutine.

In the dosage forms of the present invention, the drug can alternatively selected from the group consisting of adrenergic agents such as salts of ephedrine, desoxyephedrine, phenylephrine, epinephrine, salbutamol, terbutaline and the like; cholinergic agents such as salts of physostigmine, neostigmine and the like; curariform agents such as salts of chlorisondamine and the like; antidepressants like salts of amitriptyline, nortriptyline, and the like; and cardioactive agents such as salts of verapamil, diltiazem, gallapomil, cinnarizine, propranolol, metoprolol, nadolol, clonidine, methyldopa, nifedipine or salts thereof. The drug can also be selected from the group consisting of antimalarials such as chloroquine and the like; analgesics such as propoxyphene, meperidine and the like; beta-blockers such as propranolol, metoprolol, atenolol, labetolol, timolol and pindolol; antimicrobial agents such as cephalexin, cefaclor, cefadroxil, cefuroxime, cefuroxime axetil, erythromycin, penicillin, 7-[D-(aminophenylacetyl)amino]-3-chloro-8-oxo-1-azabicyclo[4.2.0]oct-2-ene-2-carboxylic acid, also known as loracarbef, 7-[amino[3-[(methylsulfonyl)amino]-phenyl]acetyl]amino]amino]-3-chloro-8-oxo-1-azabicyclo[4.2.0]-oct-2-ene-2-carboxylic acid, and 7-[D-amino[3-[(ethylsulfonyl)amino]phenyl]acetyl]amino-3-chloro-8-oxo-1-azabicyclo[4.2.0]oct-2-ene-2-carboxylic acid; tranquilizers such as diazepam, chlordiazepoxide and oxazepam; anticonvulsants; antihistamines such as salts of diphenhydramine, chlorpheniramine, dimenhydrinate, tripelennamine, perphenazine, brompheniramine, chlorprophenazine, chlorprophenpyridamine and the like, antinauseants; antiemetics; tranquilizers and muscle relaxants such as salts of fluphenazine, thioridazine, trifluoperazine, chlorpromazine, triflupromazine and the like; anti-inflammatory substances; psychotropics; antimanics; stimulants; decongestants; antianginal drugs; vasodilators; antiarrhythmics; vasoconstrictors; migraine treatments; diuretics; antispasmodic agents such as salts of atropine, methantheline, papaverine and the like antiasthmatics; anti-parkinson agents; expectorants; cough suppressants; mucolytics; vitamins; mineral and nutritional additives.

The dosage forms of the present invention preferably further comprises a floating layer. The floating layer can comprise at least one ingredient having a true density, which is less than density of water. Preferably, the floating layer comprise a component selected from the group consisting of methacrylic acid copolymers (e.g. Eudragit L, Eudragit E, Eudragit S100), stearyl alcohol, hydrogenated vegetable oil (e.g. Lubritab), glyceryl monooleate, glyceryl monostearate, glyceryl behenate, glyceryl palmitostearate, medium chain triglycerides, lecithin, and stearic acid, or alternatively being made of at least one excipient having a true density higher than water, but that when compressed at low pressure such that the density of the resulting tablet is less than water, while providing high tablet hardness and integrity, such as Kollidon SR. The floating layer can be present in the dosage form in an amount of about 20 to about 80% by weight (preferably about 30 to about 60% by weight) based on the total weight of the dosage form.

In the dosage forms of the present invention, the organic acid or salt thereof can be present in the dosage form in an amount of about 5 to about 50% by weight (preferably about 10 to about 35% by weight) based on the total weight of the dosage form. The organic acid is preferably sorbic acid. The drug can be present in the dosage form in amount of about 5 to about 70% by weight (preferably about 10 to about 50% by weight) based on the total weight of the dosage form. Preferably, the dosage form further comprises at least one pharmaceutical acceptable carrier. The pharmaceutical acceptable carrier can be present in the dosage form in an amount of about 30 to about 90% by weight (preferably about 50 to about 70% by weight) based on the total weight of the dosage form.

The dosage forms of the present invention can release about 10 to about 75% (preferably about 20 to about 60%) of the total amount of the drug, into the mammal stomach.

The dosage forms of the present invention can release about 25 to about 75% (preferably about 35 to about 65%) of the total amount of the drug, into the mammal proximal intestine.

The dosage forms of the present invention can release less than about 30% (preferably less than about 10%) of the total amount of the drug, into the mammal colon.

The dosage forms of the present invention are particularly useful for human. The dosage forms of the present invention are also preferably once-a-day dosage forms or twice-daily dosage forms. Preferably, the dosage forms are pH insensitive. They can have a substantially constant dissolution rate for a period of at least 1 to 8 hours when administered to the mammal. Alternatively, they can have, one hour after administration, a substantially constant dissolution rate over a period of at least 1 to 7 hours.

The dosage forms can have a diameter of about 9 mm to about 14 mm, and preferably of about 10 mm to about 12 mm. The dosage forms can have a density of about 0.7 g/mL to about 1.0 g/mL, and preferably of about 0.7 g/mL to about 0.95 g/mL.

In accordance with a one embodiment of the present invention there is provided a once-a-day sustained-release oral dosage form comprising:

a first layer including a drug and sorbic acid or a salt thereof; and

a second layer including a floating agent,

wherein the sorbic acid or salt thereof reduces the dissolution rate of the drug at a pH of about 1.0 to about 3.5, and enhances the dissolution rate of the drug at a pH of about 4.0 to about 7.0.

In accordance with another embodiment of the present invention there is provided a once-a-day sustained-release oral dosage form comprising:

a first layer including a drug and sorbic acid or a salt thereof; and

a second layer including a floating agent,

wherein the sorbic acid or salt thereof is effective for maintaining the dissolution rate of the drug at a value of about 5% by weight per hour to about 50% by weight per hour, based on the total weight of the drug included in the dosage form, when the dosage form is at a pH of about 4.0 to about 7.0.

In accordance with another embodiment of the present invention there is provided a once-a-day sustained-release oral dosage form comprising:

a first layer including a drug and sorbic acid or a salt thereof; and

a second layer including a floating agent,

wherein the sorbic acid or salt thereof is effective for maintaining the dissolution rate of the drug at a value of about 10% by weight per hour to about 60% by weight per hour, based on the total weight of the drug included in the dosage form, when the dosage form is at a pH of about 1.0 to about 3.5.

In accordance with another embodiment of the present invention there is provided a once-a-day sustained-release oral dosage form comprising:

a first layer including a drug and sorbic acid; and

a second layer including a floating agent,

wherein the dosage form releases no more than 60% of the total amount of the drug within a first hour, the remaining amount of the drug being released over a period of time of about 5 to about 8 hours subsequent to the first hour.

In accordance with another embodiment of the present invention there is provided a once-a-day sustained-release oral dosage form comprising:

a first layer including a drug and sorbic acid; and

a second layer including a floating agent,

wherein the drug in the dosage form has a dissolution rate of about 10% by weight per hour to about 60% by weight per hour when being at a pH of about 1.0 to about 3.5, and a dissolution rate of about 5% by weight per hour to about 50% by weight per hour when being at a pH of about 4.0 to about 7.0.

The dosage forms of the present invention can be used in the treatment of acne, preferably when the drug is minocycline. They can also be used in the treatment of infection by anthrax. When used for treating anthrax, the drug in the dosage form of the present invention is preferably minocycline, doxycycline, or a mixture thereof. Alternatively, the dosages forms of the present invention can be used for the treatment of irritable bowel syndrome. In such a case, the drug is preferably trimebutine.

In accordance with another aspect of the present invention there is provided a kit for treating acne comprising a dosage form as defined in the present invention and instructions for using the dosage form.

In accordance with another aspect of the present invention there is provided a kit for treating infection by anthrax comprising a dosage form as defined in the present invention and instructions for using the dosage form.

In accordance with another aspect of the present invention there is provided a kit for treating irritable bowel syndrome comprising a dosage form as defined in the present invention and instructions for using the dosage form.

In accordance with another aspect of the present invention there is provided a method for treating acne comprising the step of administering to a patient suffering from this disease a dosage form as defined in the present invention. The dosage form is preferably administered to a patient once-a-day. The drug in such a dosage form is preferably minocycline.

In accordance with another aspect of the present invention there is provided a method for treating infection by anthrax comprising the step of administering to a patient suffering from this disease a dosage form as defined in the present invention. The drug in such a dosage form is preferably minocycline or doxycycline.

In accordance with another aspect of the present invention there is provided a method for treating irritable bowel syndrome comprising the step of administering to a patient suffering from this disease a dosage form as defined in the present invention. The drug in such a dosage form is preferably trimebutine.

In accordance with another aspect of the present invention there is provided the use of sorbic acid or a salt thereof for controlling the dissolution rate of a drug in a dosage form comprising the drug and the sorbic acid or salt thereof, wherein the sorbic or salt thereof acid reduces the dissolution rate of the drug at a pH of about 1.0 to about 3.5, and enhances the dissolution rate of the drug at a pH of about 4.0 to about 7.0.

In accordance with another aspect of the present invention there is provided the use of sorbic acid or a salt thereof for controlling the dissolution rate of a drug in a dosage form comprising the drug and the sorbic acid or salt thereof, wherein the sorbic acid or salt thereof is effective for maintaining the dissolution rate of the drug at a value of about 5% by weight per hour to about 50% by weight per hour, based on the total weight of the drug included in the dosage form, when the dosage form is an environment at pH of about 4.0 to about 7.0.

In accordance with another aspect of the present invention there is provided the use of sorbic acid or a salt thereof as a dissolution regulator in the preparation of a dosage form for releasing a pH-dependent solubility drug.

In accordance with another aspect of the present invention there is provided the use of sorbic acid or a salt thereof as a dissolution regulator in the preparation of a dosage form for releasing a pH-dependent solubility drug, for providing to the drug a dissolution rate of about 5% by weight per hour to about 50% by weight per hour, based on the total weight of the drug included in the dosage form, when the dosage form is in an environment at a pH of about 4.0 to about 7.0.

In accordance with another aspect of the present invention there is provided the use of sorbic acid or a salt thereof as a dissolution regulator in the preparation of a dosage form for releasing a pH-dependent solubility drug, for providing to the dosage form a dissolution rate which is substantially pH-insensitive when pH is about 1.0 to about 7.0.

In accordance with another aspect of the present invention there is provided a method for regulating or controlling the dissolution rate of a pH-dependent solubility drug comprising the step of preparing a dosage form including the drug and sorbic acid or a salt thereof.

In accordance with another aspect of the present invention there is provided a method for regulating or controlling the dissolution rate of a pH-dependent solubility drug in such a manner that the drug has a dissolution rate which is substantially the same when submitted to a pH of about 1.0 to about 3.5 and when submitted to a pH of about 4.0 to about 7.0, the method comprising the step of preparing a dosage form including the drug and sorbic acid or a salt thereof.

In accordance with another aspect of the present invention there is provided a method for enhancing absorption of a drug at the level of proximal intestine, the method comprising the step of administering to a patient an oral dosage form including the drug and sorbic acid or a salt thereof.

In accordance with another aspect of the present invention there is provided a method for enhancing delivery of a drug to stomach and proximal intestine of a patient while minimizing delivery to distal intestine and colon of the patient, the method comprising orally administering to the patient a sustained-release oral dosage form comprised of a drug and sorbic acid or salt thereof, wherein sorbic acid or salt thereof reduces the dissolution rate of the drug when the dosage form is within stomach of the patient at a pH of about 1.0 to about 3.5, and wherein sorbic acid enhances the dissolution rate of the drug when the drug is within proximal intestine of the patient at a pH of about 4.0 to about 7.0.

In accordance with another aspect of the present invention there is provided a method for enhancing delivery of a drug to stomach and proximal intestine of a patient while minimizing delivery to distal intestine and colon of the patient, the method comprising orally administering to the patient an oral dosage form as defined in the present invention.

In accordance with another embodiment of the present invention there is provided an oral dosage form for treating acne. The dosage form comprises

minocycline and an organic acid selected form the group consisting of fumaric acid, adipic acid, glutamic acid, sorbic acid, benzoic acid and salts thereof,

wherein the dosage form is suitable for treating acne by means of a once-a-day administration. The organic acid is preferably sorbic acid.

In accordance with another embodiment of the present invention there is provided an oral dosage form for treating acne. The dosage form comprises:

a first layer including minocycline and an organic acid selected form the group consisting of fumaric acid, adipic acid glutamic acid, sorbic acid, and salts thereof,

a second layer including a floating agent,

wherein the dosage form is suitable for treating acne by means of a once-a-day administration. The organic acid is preferably sorbic acid.

In accordance with another embodiment of the present invention there is provided a controlled-release oral dosage form for releasing a drug according to a drug release profile as defined in FIG. 3.

In accordance with another embodiment of the present invention there is provided a controlled-release oral dosage form for releasing a drug, wherein the dosage form allows to the drug a controlled-release permitting to the drug to have a plasma concentration as defined in FIG. 1.

In accordance with another embodiment of the present invention there is provided a controlled-release oral dosage form for releasing a drug, the dosage form being characterized in that it permits to the drug a release as defined in FIG. 3.

In accordance with another embodiment of the present invention there is provided a controlled-release oral dosage form for releasing a drug, the dosage being characterized in that it permits to the drug to have a plasma concentration as defined in FIG. 1.

The person skilled in the art would understand that the preferred embodiments with respect to the dosage forms of the present invention can also apply to the kit, methods and uses of the present invention. It should also be understood that the dosage forms of the invention are all provided to patients in therapeutically effective amounts.

### BRIEF DESCRIPTION OF THE DRAWINGS

Several features and advantages of the present invention will become more readily apparent from the following drawings which represent in a non-limitative manner preferred embodiments of the invention.

FIG. 1 represents a comparison between plasmatic concentration curves of Minocin™ immediate release twice daily 50 mg and a 100 mg control-release dosage form according to a preferred embodiment of the present invention, in which the drug is minocycline;

FIG. 2 represents a dosage form according to another preferred embodiment of the invention, wherein the dosage form has a bilayer tablet configuration;

FIG. 3 represents a drug release profile, at various pH, of a bilayer dosage form according to another preferred embodiment of the present invention, wherein the drug is minocycline;

FIG. 4 represents a dosage form according to another preferred embodiment of the invention, wherein the dosage form has a single layer configuration;

FIG. 5 represents a drug release profile, at various pH, of a single layer dosage form according to another preferred embodiment of the present invention, wherein the drug is trimebutine; and

FIG. 6 represents a drug release profile, at various pH, of a single layer dosage form according to another preferred embodiment of the present invention, wherein the drug is minocycline (200 mg).

### DETAILED DESCRIPTION OF PREFERRED EMBODIMENTS OF THE INVENTION

The present invention will be more readily understood by referring to the following example, which represents in a non-limitative manner preferred embodiments.

### EXAMPLE 1

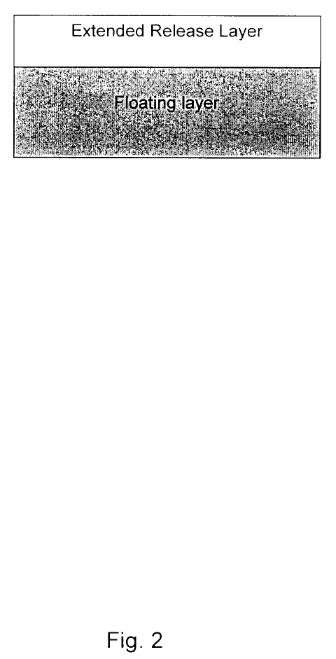

A controlled release dosage form was prepared according to following general method. The ingredients and their proportions are shown in Table 1. The dosage form of Example 1, aminocycline tablet, is in fact a bilayer tablet as shown in FIG. 2. It comprises a release layer including minocycline and a floating layer.

The minocycline release layer is manufactured as follows. Minocycline HCl, sorbic acid, and HPMC are granulated with water in a high shear granulator. The granules are then dried in a fluid bed dryer, and sized. Kollidon SR is added, and then the blend is lubricated with PRUV. The floating layer is prepared by first blending Kollidon SR and Lubritab. Silicon dioxide is incorporated, and the blend is lubricated by incorporating PRUV.

The tablet is formed onto a single punch Manesty F3 press by first introducing 270 mg of the floating layer introduced into a 12.0 mm round die (concave tooling punch). The floating layer is tamped slightly by lowering upper punch. The release layer (325 mg) is introduced into the die on top of the floating layer, and a full compression cycle is performed. The thickness of the tablet is in the range 6.4-7.4 mm, with a hardness of 40-70N. The resulting tablet floats immediately upon contact with water.

TABLE 1
Formulation of a minocycline bilayer tablet

| | | | | |
|---|---|---|---|---|
| Item | Ingredients/ | Quantity | | Quantity |
| # | Specifications | per Batch g | % | per/tablet mg |

Release layer

| | | | | |
|---|---|---|---|---|
| | Total | 1462.763 | 100.00 | 325.0585 |
| 1 | Minocycline HCl, USP* | 519.640* | 35.525 | 115.4757* |
| 2 | Kollidon SR, EP | 285.795 | 19.538 | 63.510 |
| 3 | Hydroxypropyl Methycellulose | 285.795 | 19.538 | 63.510 |
| | (Methocel E5 Premium LV), USP | | | |
| 4 | Sorbic Acid, pulverized, NF | 364.388 | 24.911 | 80.975 |
| 5 | Sodium Stearyl Fumarate (PRUV), NF | 7.145 | 0.488 | 1.5878 |
| 6 | Distilled Water USP** | 105.284** | N/A | N/A |
| | Total | 1462.763 | 100.00 | 325.0585 |

Floating layer

| | | | | |
|---|---|---|---|---|
| 7 | Kollidon SR, EP | 448.200 | 83.00 | 224.100 |
| 8 | Hydogenated Vegetable oil, type 1 | 85.320 | 15.80 | 42.660 |
| | (Lubritab), USP | | | |
| 9 | Colloidal Silicon Dioxide (Cab-O-Sil | 2.700 | 0.50 | 1.350 |
| | M5P), USP | | | |
| 10 | Sodium Stearyl Fumarate (PRUV), NF | 3.780 | 0.70 | 1.890 |
| | Total | 540.00 | 100.00 | 270.00 |

*At 6.5% humidity and 100% potency, equivalent to 100 mg Minocycline base
**Removed during the process

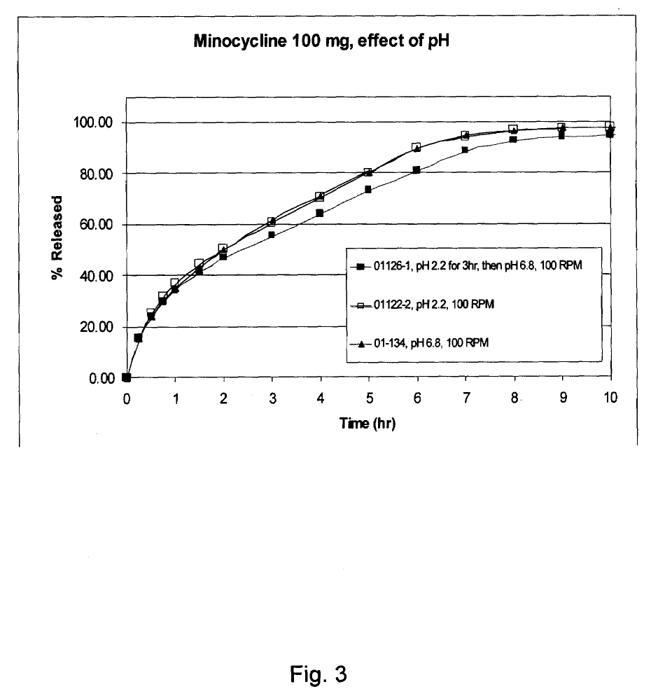

The dosage form of Example 1, as shown in FIG. 3, shows a release profile that has a relative insensitivity to different pH treatments in the course of dissolution. In fact, it can be seen from FIG. 3 that the dosage form of Example 1 is pH-insensitive.

In Example 1, a floating device (or floating layer) of relatively large diameter is added so as to keep the tablet for an extended period of time into a fed stomach. The floating object preferably remains physically intact (no extensive erosion with time), and penetration of water is preferably kept to a minimum. The air present in the tablet can help to keep the device floating. In the present invention, minocycline is preferably released from a compartment of the tablet that is separated from the floating device or layer. The floating and release layers or compartments are thus formulated into a bilayer tablet, as illustrated in FIG. 2, and which is preferably a round, bi-concave tablet.

As shown in FIG. 3, close to 40% of drug is released from the minocycline layer within the first hour. Then, the remaining dose is released over the next 6 to 8 hours i.e. 6 to 8 hours later. This release profile is somewhat peculiar, as the formulation releases rapidly initially, followed by a somewhat linear release thereafter. This is caused by the particular nature and combination of the various ingredients.

The floating layer maintains the tablet as long as possible in the stomach. A floating tablet of large diameter increases the overall residence time of tablets in fed subject (Hou et al. Critical Reviews™ in Therapeutic Drug Carrier Systems, 20(6), 461-497, 2003). Since the absorption window of minocycline is substantially limited to the proximal portion of the intestine, the longer the tablet is retained in the stomach, the longer it take for the tablet to reach intestinal regions of lesser absorption. A longer transit time can be used advantageously to stretch absorption time and obtained an extended plasmatic plateau.

Since the dosage form remains in the stomach for an extended period of time, thus at relatively low pH values of around 1.0 to 3.5 for a fed stomach, and then be transferred in the small intestine at a pH range of 5 to 7, minocycline solubility varies considerably with localization in the gastro-intestinal tract. The previously discussed figures show that sorbic acid, at low pH, reduces the solubility rate of minocycline and that at higher pH, it enhances the solubility rate of minocycline. This is a considerable advantage over the prior art minocycline dosage forms, which quickly release the drug at low pH. Moreover, in the prior art minocycline dosage forms, the release is slowed down dramatically upon transfer to neutral or higher pH. In fact, minocycline HCl has a solubility greater than 500 mg/mL at a pH of about 1.0 and it has a solubility of about 52 mg/mL at a pH of about 6.7. In the present invention, the differential solubility of minocycline HCl is compensated by sorbic acid, which has a solubility profile that is the reverse of that of minocycline HCl. The compound used in example 1 is sorbic acid. Sorbic acid has a solubility of about 2.5 mg/mL at a pH lower than 4.8, and it has solubility of about 333 mg/mL at a pH higher than 4.8.

In the tablet of Example 1 at low pH, as the solubility of minocycline HCl is relatively higher, the solubility of sorbic acid is minimal. Under this pH condition, sorbic acid acts as a barrier to the dissolution of highly soluble minocycline HCl. At higher pH, however, sorbic acid becomes readily soluble, and generates pores into the tablet which enhances the solubilisation of minocycline HCl. The lower solubility of minocycline HCl at higher pH is therefore compensated by the increased afflux of water within the tablet as sorbic acid is leached away. Kollidon SR act as a long-acting agent preserving the integrity of the tablet and opposing the rapid swelling of Methocel E5 by incoming water. Methocell E5 acts as a gelifying agent which also controls the inward movement of water, and act as a slow disintegration agent. It also controls the initial burst of minocycline. Kollidon SR further gives a tablet with high hardness at low compression force. That allows compression of the tablet at a relatively low density which guarantees the floatability of tablet. Other low molecular weight organic acids with solubility properties similar to sorbic acid and which can alternatively be used be used in the dosage form. These acids can be fumaric acid, adipic acid, benzoic acid and glutamic acid. Other low molecular weight organic acids such as citric acid and tartaric acid could also be used.

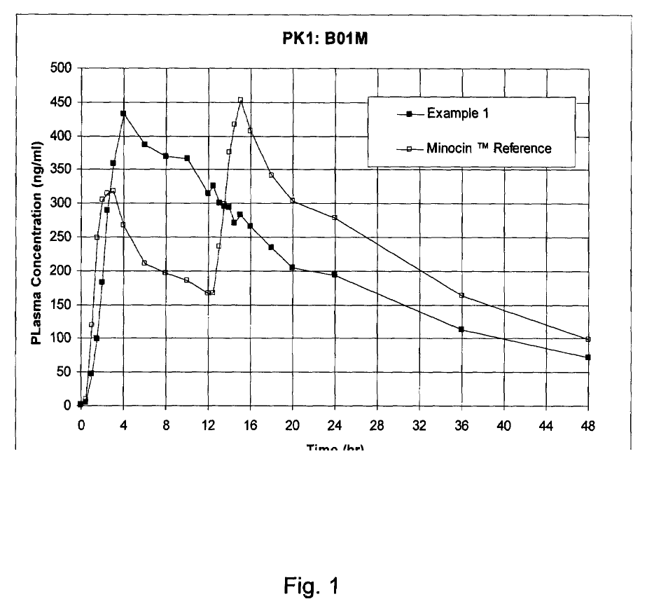

Given the relatively long plasma half-life of 11 to 26 hours for minocycline, the extended residence time provided by a floating layer is found to provide a satisfactory pharmacokinetic profile, as shown in FIG. 1. FIG. 1 shows the in vivo plasmatic concentration profiles. FIG. 1 also contains the plasmatic profile for immediate release twice daily 50 mg tablet.

As it can be seen from FIG. 1 and Table 2, the dosage of Example 1 is more favorable than Minocin™.

TABLE 2
Comparison of bioequivalents data for Minocin ™ and
the dosage form of Example 1

| | | | % RATIO OF | 90% |
|---|---|---|---|---|
| | | REFERENCE | GEOMETRIC | CONFIDENCE |
| PARAMETER | EXAMPLE 1 | Minocin ™ | MEANS | INTERVAL |
| AUC_{0-T} | 9222.47 | 10418.32 | 88.52 | 80.99-96.76 |
| (ng · h/mL) | 9640.19 (31.27) | 10608.8 (20.89) | — | — |
| AUC_{0-X (0-24)} | 6453.71 | 6287.62 | 102.64 | 95.30-110.55 |
| (ng · h/mL) | 6686.69 (27.09) | 6376.77 (17.74) | — | — |
| AUC_{0-∞} | 10932.9 | 12805.8 | 85.37 | 77.15-94.48 |
| (ng · h/mL) | 11655.5 (39.25) | 13277.0 (31.14) | — | — |
| Cₘₐₓ | 470.55 | 478.60 | 98.32 | 89.85-107.59 |
| (ng/mL) | 481.42 (22.14) | 489.75 (22.26) | | |
| Tₘₐₓ | — | — | — | — |
| (h) | 7.08 (56.22) | 13.95 (25.12) | — | — |

FIG. 1 shows plasmatic concentration of drug in blood circulation for the once-a-day dosage form of Example 1 as opposed to the twice-a-day reference formulation Minocin™. Table 2 provides numerical data describing and summarizing the critical information that can be extracted from the curves in FIG. 1. Of considerable importance is the comparison of the area under the curve (AUC) for the two products and the ratio of the geometric means for the AUC for the test and reference products. For example, the % ratio of the geometric means for the AUC over 24 hr is 102.64%, and is 88.52 over 48 hrs, indicating the test and reference products are bioequivalent. The tablet also indicate that the maximum concentration achieve is similar in either cases. Tmax is somewhat different, as can be expected, but that has no impact on bioavailability properties.

The person skilled in the art would thus recognize the several advantages of the dosage form of Example 1 over Minocin™. Firstly, the dosage form of Example 1 is a once-a-day dosage form as opposed to Minocin™, which has to be taken twice a day. As previously mentioned, the dosage form of Example 1 is bioequivalent to Minocin™, i.e. a similar amount of drug is found into the blood circulation when the dosage form of Example 1 or Minocin™ are taken. However, patient takes only one tablet of the dosage form of Example 1 per day instead of two tablets Minocin™ per day. Besides being more practical, taking one tablet per day results in better patient compliance to treatment than having to take two Minocin™ tablet per day.

The dosage form of Example 1 shows a quick unset of plasmatic concentration, almost as quick as Minocin™, and then shows a sustained plasmatic concentration. On the other hand, Minocin™ provides a marked fluctuation in blood concentration over a 24 hr cycle. The variation in blood concentration is lessened with the dosage form of Example 1 as compared to Minocin. Such a fact can be clearly seen form FIG. 1 since the dosage form of Example 1 provides essentially one peak and one valley per 24 hrs, whereas Minocin™ provides two peaks and two valleys per day.

Many other different dosages can be prepared in accordance with the present invention, ranging from 20 mg to 300 mg.

### EXAMPLE 2

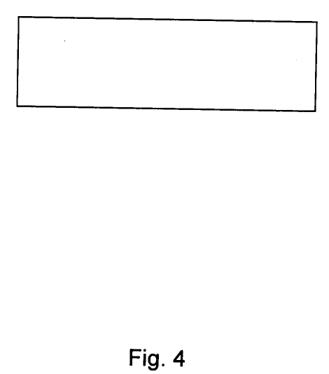

A controlled release dosage form was prepared according to following general method. The ingredients and their proportions are shown in Table 3. The dosage form of Example 2, a trimebutine tablet, is in fact a single layer tablet as shown in FIG. 4.

TABLE 3
Formulation of a trimebutine single layer tablet

| Ingredient | mg/tab | W/W % |
|---|---|---|
| Trimebutine Maleate | 300.0 | 45.6 |
| Hydroxypropyl Methycellulose (Methocel | 106.4 | 16.2 |
| E5 Premium LV) | | |
| Sorbic Acid | 135.6 | 20.6 |
| Propyl gallate | 0.08 | 0.012 |
| Kollidon SR | 106.4 | 16.2 |
| Colloidal silicon dioxide | 3.3 | 0.5 |
| Sodium Stearyl Fumarate (PRUV) | 6.6 | 1.0 |
| Total for tablet | 658.4 | 100.00 |

The tablet is manufactured as follows. Trimebutine maleate, sorbic acid, propyl gallate, and HPMC are granulated with water in a high shear granulator. The granules are then dried in a fluid bed dryer, and sized. Kollidon SR is added, and then the blend is lubricated with Colloidal silicon dioxide and PRUV.

The tablet is formed onto a single punch Manesty F3 press using a 12.0 mm round die (concave tooling punch). The thickness of the tablet is in the range 6.0-6.5, with a hardness of over 90N. The resulting tablet is not a floating tablet.

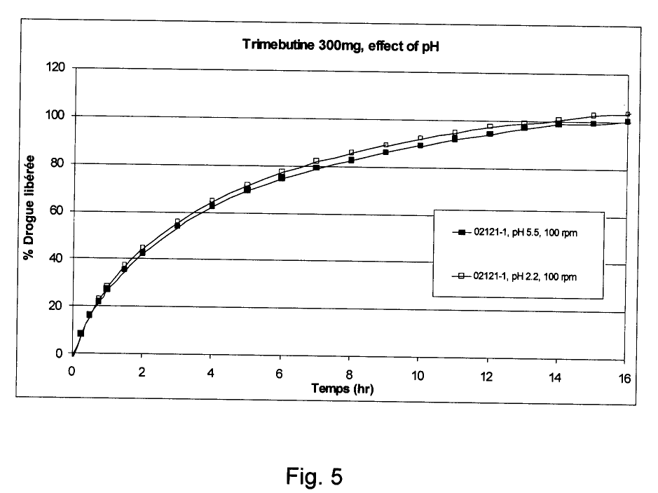

The dosage form of Example 2, as shown in FIG. 5, shows a release profile that has a relative insensitivity to different pH treatments in the course of dissolution. In fact, it can be seen from FIG. 5 that the dosage form of Example 2 is pH-insensitive.

In Example 2, the retention of the tablet for an extended period of time into a fed stomach is essentially caused by the relatively large diameter of the tablet. The tablet has been formulated to remains physically intact with limited erosion for the initial few hours.

As shown in FIG. 5, close to 40% of drug is released from the tablet within the first 2 hours. Then, the remaining dose is released over the next 8 to 10 hrs. This release profile is typical of a first order release, whereas Example 1, FIG. 3, was more typical of a zero-order release.

Trimebutine has a solubility of about 11 mg/ml at a pH of 2, while its solubility at a pH of 5.5 is 1 mg/ml. Thus, the solubility of trimebutine varies considerably with localization in the gastrointestinal tract. The previously discussed figures show that the formulation incorporating sorbic acid compensates the differential solubility of trimebutine as a function of pH.

### EXAMPLE 3

A controlled release dosage form was prepared according to following general method. The ingredients and their proportions are shown in Table 4. The dosage form of Example 3, a 200 mg minocycline tablet, is in fact a single layer tablet as shown in FIG. 4.

TABLE 4
Formulation of Minocycline 200 mg single layer tablet

| Ingredient | mg/tab | W/W % |
|---|---|---|
| Minocycline HCl | 231.0* | 37.4 |
| Hydroxypropyl Methycellulose (Methocel | 127.0 | 20.5 |
| E5 Premium LV) | | |
| Sorbic Acid | 162.0 | 26.2 |
| Propyl gallate | 0.16 | 0.03 |
| Kollidon SR | 94.9 | 15.4 |
| Sodium Stearyl Fumarate (PRUV) | 3.1 | 0.5 |
| Total for tablet | 618.2 | 100.0 |

*considering humidity and potency, equivalent to 200 mg Minocycline

The tablet is manufactured as follows. Minocycline HCl, sorbic acid, propyl gallate, and HPMC are granulated with water in a high shear granulator. The granules are then dried in a fluid bed dryer, and sized. Kollidon SR is added, and then the blend is lubricated with PRUV.

The tablet is formed onto a single punch Manesty F3 press using a 12.0 mm round die (concave tooling punch). The thickness of the tablet is in the range 6.0-6.5, with a hardness of over 110N. The resulting tablet is not a floating tablet.

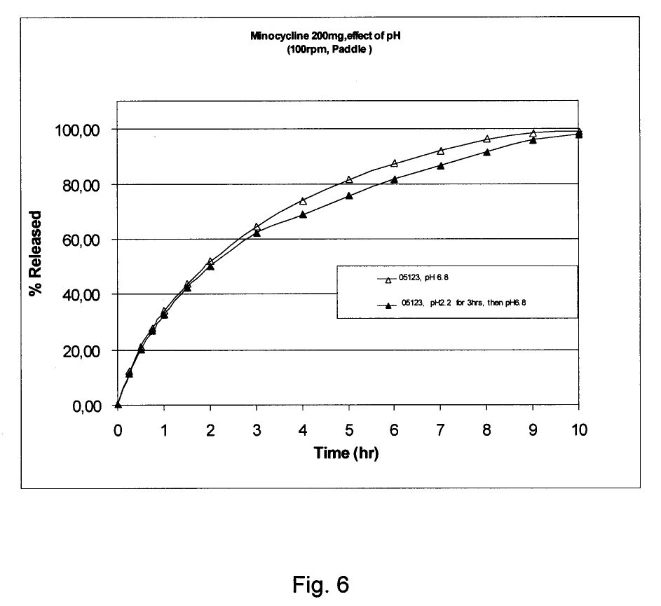

The dosage form of Example 3, as shown in FIG. 6, shows a release profile that has a relative insensitivity to different pH treatments in the course of dissolution. In fact, it can be seen from FIG. 5 that the dosage form of Example 2 is pH-insensitive.

In Example 3, the retention of the tablet for an extended period of time into a fed stomach is essentially caused by the relatively large diameter of the tablet. The tablet has been formulated to remains physically intact with limited erosion for the initial few hours.

As shown in FIG. 6, close to 35% of drug is released from the tablet within the first hour. Then, the remaining dose is released over the next 6 to 8 hrs. This release profile is closer to that of a first order release, whereas in Example 1, FIG. 3, was more typical of a zero-order release.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

As previously indicated, a major difficulty encountered in drug delivery is the variation of the solubility of a drug as a function of pH. The difficulty of developing a controlled-release dosage form with molecules having a pH-dependant solubility has been the subject of many publications.

Some authors have used high molecular weight polyacids to offset the reduced solubility of the drug as pH increases. The polyacids become soluble at higher pH, which triggers erosion of the system. The rapid erosion of the system counter-balances the decreased solubility of the drug.

WO 97/18814 describes a formulation comprising a low molecular weight polyethylene oxide, HPMC and one or more enteric polymers such as methacrylic acid copolymers. With compounds being more soluble at low pH value, the drug release from the tablet in the stomach is reduced by the enteric polymer. However, as the device travel along the gastrointestinal tract and the pH increases, the erosion rate increases to compensate for the reduced compound solubility.

U.S. Pat. No. 4,968,508 describes a formulation comprising a hydrophilic polymer (e.g. HPMC) and an enteric polymer (e.g. Eudragit™ L-100-55). In acidic medium, the enteric polymer is insoluble and acts as a part of the matrix and thus contributes to the retardation of the drug release. At higher pH-values, the enteric polymer dissolves and thus increases the permeability of the dosage form.

U.S. Pat. No. 4,792,452 describes a formulation containing an anionic (sodium alginate) and non-ionic polymer (HPMC). At low pH values, sodium alginate precipitates in the hydrated gel layer as alginic acid and provides resistance against erosion. At higher pH-values, the alginate forms a soluble salt, thus providing less resistance to erosion. The release mechanism changes from predominantly diffusion-controlled to predominantly erosion-controlled. The resulting higher permeability of the gel layer at high pH-value compensates the lower drug solubility, leading to a constant drug release rates at different pH-values.

1. A controlled-release oral dosage form for administration to a mammal, said dosage form comprising:
a drug and an organic acid selected form the group consisting of fumaric acid, adipic acid, glutamic acid, sorbic acid, benzoic acid and salts thereof,
wherein said dosage form releases no more than 60% of the total amount of the drug within about a first hour, the remaining amount of said drug being released over a period of time of about 5 to about 10 hours subsequent to the first hour.

2. The dosage form of claim 1, wherein said drug is a pH-dependent solubility drug, which is substantially soluble at a first pH of about 1.0 to about 3.5 and substantially less soluble at a pH of about 4.0 to 7.0 than at said first pH.

3. The dosage form of claim 1, wherein said drug is selected from the group consisting of doxycycline, tetracycline, oxytetracycline, minocycline, chlortetracycline, trimebutine, demeclocycline and salts thereof.

4. The dosage form of claim 2, wherein said drug is minocycline hydrochloride.

5. The dosage form of claim 2, wherein said drug is trimebutine maleate.

6. The dosage form of any one of claim 1, wherein said dosage further comprises a floating layer in an amount of about 20 to about 80% by weight based on the total weight of said dosage form.

7. The dosage form of claim 6, wherein said floating layer comprises at least one ingredient having a true density, which is less than density of water, such ingredient being selected from the group consisting of methacrylic acid copolymers (e.g. Eudragit™ L, Eudragit™ E, Eudragit™ S100), stearyl alcohol, hydrogenated vegetable oil (e.g. Lubritab™), glyceryl monooleate, glyceryl monostearate, glyceryl behenate, glyceryl palmitostearate, medium chain triglycerides, lecithin, and stearic acid, or alternatively being made of at least one excipient having a true density higher than water, but that when compressed at low pressure such that the density of the resulting tablet is less than water, while providing high tablet hardness and integrity, such as Kollidon™ SR.

8. The dosage form of claim 6, wherein said floating layer comprises Kollidon SR and Lubritab.

9. The dosage form of claim 1, wherein said sorbic acid or salt thereof is present in said dosage form in an amount of about 5 to about 50% by weight based on the total weight of said dosage form.

10. (canceled)

11. The dosage form of claim 1, wherein said drug is present in said dosage form in amount of about 5 to about 70% by weight based on the total weight of said dosage form.

12. (canceled)

13. The dosage form of claim 1, wherein said dosage form further comprises at least one pharmaceutical acceptable carrier in an amount of about 30 to about 90% by weight based on the total weight of said dosage form.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The dosage form of claim 1, wherein said dosage form has a density of about 0.7 g/mL to about 1.0 g/mL.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. A controlled-release oral dosage form for releasing a drug according to a drug release profile as defined in FIG. 3, FIG. 5 or FIG. 6.

54. (canceled)

55. (canceled)

56. (canceled)

57. (canceled)

58. (canceled)

59. (canceled)

60. (canceled)

61. (canceled)

62. (canceled)

63. A method for treating acne comprising the step of administering to a patient suffering from this disease a dosage form as defined in claim 1.

64. The method of claim 63, wherein said dosage form is administered to a patient once-a-day.

65. (canceled)

66. A method for treating irritable bowel syndrome comprising the step of administering to a patient suffering from this disease a dosage form as defined in claim 1.

67. (canceled)

68. (canceled)

69. (canceled)

70. (canceled)

71. (canceled)

72. (canceled)

73. (canceled)

74. A method for enhancing absorption of a drug at the level of proximal intestine, said method comprising the step of administering to a patient an oral dosage form including said drug and an organic acid selected form the group consisting of fumaric acid, adipic acid, glutamic acid, sorbic acid, benzoic acid and salts thereof.

75. The method of claim 74, wherein said drug is minocycline.

76. (canceled)

77. (canceled)
